(19) United States
(12) Patent Application Publication
(10) Pub. No.: US 2005/0284797 A1
(43) Pub. Date: Dec. 29, 2005

(54) Integrated plant process to produce high molecular weight basestocks from fischer-tropsch wax

(76) Inventors: William Berlin Genetti, Baton Rouge, LA (US); Adeana Richelle Bishop, Baton Rouge, LA (US); Louis Francis Burns, Baton Rouge, LA (US); Loren Leon Ansell, Baton Rouge, LA (US); Jack Wayne Johnson, Clinton, NJ (US); Charles Harrison Mauldin, Baton Rouge, LA (US)

Correspondence Address: ExxonMobil Research and Engineering Company, P. O. Box 900, Annandale, NJ, 08801-0900, US

(21) Appl. No.: 11/109,122

(22) Filed: Apr. 19, 2005

Related U.S. Application Data
(60) Provisional application No. 60/583,081, filed on Jun. 25, 2004.

(51) Int. Cl. C07C 27/26; C07B 63/02

(52) U.S. Cl. 208/115; 518/726

(57) ABSTRACT

High viscosity lube base oils are produced from syngas by converting syngas under Fischer-Tropsch reaction conditions which are sufficient to produce hydrocarbon products containing greater than 20 lbs of 700° F.+(371° C.+) product per 100 lbs of CO converted. A 450° F.+(232° C.+) cut is separated from the hydrocarbon products and catalytically hydroisomerized and distilled to provide high viscosity lube base oils.

This application claims the benefit of U.S. Ser. No. 60/583,081 filed Jun. 25, 2004.

### FIELD OF INVENTION

The present invention is broadly concerned with the preparation of lube base oils. More particularly, the invention relates to a process for preparing high viscosity lube base oils from a Fischer-Tropsch product derived from syngas.

### BACKGROUND OF INVENTION

Hydrogenation reactions of carbon monoxide are well known. One example is the catalytic conversion of a mixture of hydrogen and carbon monoxide, i.e., syngas, to hydrocarbons via the Fischer-Tropsch process. Depending upon the catalyst and process conditions employed, a wide variety of hydrocarbon products can be obtained. The catalysts typically used include cobalt, ruthenium and iron catalysts. Cobalt and ruthenium make primarily paraffinic products, cobalt tending towards a heavier product slate, e.g., containing C₂₀+ hydrocarbons, while ruthenium tends to produce more distillate type paraffins, e.g., C₅-C₂₀ hydrocarbons. Regardless of the catalyst or conditions employed the result is the formation of at least some waxy Fischer-Tropsch products. Waxy products have poor cold flow properties limiting their value unless converted into more useable products.

Cold flow properties can be improved by increasing the branching of the Fischer-Tropsch products by subjecting the products to treatments such as hydrotreating, hydroisomerization and hydrocracking. Such treatment however tends to produce gaseous and light products that reduce the yield of more valuable products. Thus, there remains a need for maximizing lube oils, especially high viscosity lube oils, that can be obtained from Fischer-Tropsch waxes. Also it would be advantageous to integrate the formation of Fischer-Tropsch waxes with their conversion into high viscosity lube base oils. The present invention provides such a process.

### SUMMARY OF INVENTION

Broadly stated, high viscosity lube base oils are produced from syngas by converting syngas under Fischer-Tropsch reaction conditions which are sufficient to produce hydrocarbon products containing greater than 20 lbs. of 700° F.+(371° C.+) product per 100 lbs. of CO converted. A 450° F.+(232° C.+) cut is separated from the hydrocarbon products and catalytically hydroisomerized and distilled to provide high viscosity lube base oils.

In one embodiment the Fischer-Tropsch process is conducted in a slurry bubble column at temperatures no greater than 430° F. (221° C.) in the presence of a cobalt/rhenium catalyst supported on titania.

In another embodiment the Fischer-Tropsch process is conducted in the presence of a catalyst comprising cobalt on a support comprising primarily titania and a minor amount of cobalt aluminate.

Preferably the separated 450° F.+(232° C.+) cut of the hydrocarbon product is hydroisomerized in the presence of a catalyst comprising a hydrogenating metal component on a refractory oxide support.

### DETAILED DESCRIPTION OF INVENTION

The present invention provides an integrated method for producing high viscosity lube base oils from a hydrocarbon stream obtained by conducting a Fischer-Tropsch process under conditions sufficient to produce hydrocarbon products containing greater than 20 lbs of 700° F.+(371° C.+) product per 100 lbs of CO converted.

The method involves separating a 450° F.+(232° C.+) cut from the hydrocarbon products and subjecting it to a catalytic hydroisomerization step and thereafter separating a distillate and a high boiling lube cut, e.g., 700° F.+(371° C.+) or higher, of the hydroisomerized material to provide a high viscosity lube base oil. Optionally high boiling cut is dewaxed to lower the pour point, for example, to below about 0° C. Solvent or catalytic dewaxing methods in this instance may be employed.

The lube base oils obtained by the process typically have a viscosity at 100° C. of greater than 12 cSt, for example from about 13 cSt to about 40 cSt.

An advantage of the present process is the ability to provide a range of very high viscosity synthesis lubricant base stocks.

As previously stated in the practice of the present invention the Fischer-Tropsch process is conducted under conditions sufficient to produce hydrocarbon products containing greater than 20 lbs of 700° F.+(371° C.+) product per 100 lbs. of CO converted. Preferably the process is conducted under conditions to provide greater than about 24 lbs of 700° F.+(371° C.+) product per 100 lbs. of CO converted. This can be achieved by at least one of (a) the appropriate selection of process operating conditions and (b) choice of catalyst.

In the integrated process disclosed herein the Fischer-Tropsch process is conducted at temperatures no greater than 430° F. (221° C.), for example, from about 300° F. to about 430° F. (148° C. to 221° C.). Preferably the reaction is conducted at no greater than 410° F. (210° C.). Operating pressures typically are in the range of from about 10 to about 600 psia, preferably from about 250 to about 350 psia, and space velocities of about 1000 to 25,000 cc/cc/hr.

The Fischer-Tropsch process preferably is conducted in a slurry bubble column reactor. In slurry bubble column reactors catalyst particles are suspended in a liquid and gas is fed into the bottom of the reactor through a gas distributor. As the gas bubbles rise through the reactor the reactants are absorbed into the liquid and diffuse to the catalyst where they can be converted to both gaseous and liquid products. Gaseous products can be recovered at the top of the column and liquid products are recovered by passing the slurry through a filter which separates the solid catalyst from the liquid. An optimal method for operating a three phase slurry bubble column is disclosed in EP 0450860 B 1 which is incorporated herein by reference in its entirety.

Suitable Fischer-Tropsch catalysts comprise one or more Group VIII metals such as Fe, Ni, Co, and Ru on an inorganic oxide support. Additionally, the catalyst may also contain a promoter metal. One suitable catalyst for the process of the invention is cobalt promoted with rhenium supported on titania having a Re:Co weight ratio in the range of about 0.01 to 1 and containing about 2 to 50 wt % cobalt. Examples of such catalysts can be found in U.S. Pat. No. 4,568,663 (no binder); U.S. Pat. No. 4,992,406 (Al₂O₃ binder); and, U.S. Pat. No. 6,117,814 (SiO₂—Al₂O₃ binder).

Another suitable and preferred catalyst for the Fischer-Tropsch process comprises cobalt and especially cobalt and rhenium on a support comprising primarily titania and a minor amount of cobalt aluminate. In general the support will contain at least 50 wt % titania and preferably from 80 to about 97 wt % titania based on the total weight of the support. About 20 to 100 wt %, and preferably 60 to 98 wt % of the titania of the support is in the rutile crystalline phase with the balance being the anatase crystalline phase or amorphous phases. The amount of cobalt aluminate in the binder is dependent upon the amount of cobalt and aluminum compounds used in forming the support. Suffice it to say that sufficient cobalt is present in the support to provide a cobalt/aluminum atomic ratio greater than 0.25, preferably from 0.5 to 2, and more preferably about 1. Thus, at a Co/Al ratio of 0.25 about half the aluminum oxide is present as cobalt aluminate. At a Co/Al ratio of 0.5 substantially all the alumina oxide present is present as cobalt aluminate. At Co/Al ratios above 0.5 the support will contain cobalt titanate in addition to cobalt aluminate and be essentially free of alumina.

The support is typically formed by spray drying a suitable aqueous slurry of titania, alumina binder material and optionally silica binder material into a purged chamber with heated air at an outlet temperature of about 105° C. to 135° C. Spray drying produces a spherical support with a size range of about 20 to 120 microns. This spray dried support is then calcined at temperatures in the range of 400° C. to 800° C., preferably about 700° C. Next the calcined material is impregnated with an aqueous solution of a cobalt compound, preferably cobalt nitrate, in an amount sufficient to convert, upon calcination, at least part of the alumina to cobalt aluminate. Preferably sufficient cobalt compound is used to convert from 50% to 99+% of the alumina to cobalt aluminate. Therefore, the amount of cobalt compound added during the preparation of the support will correspond to an atomic ratio of Co:Al in the range of 0.25:1 to 2:1 and preferably 0.5:1 to 1:1. Indeed, it is especially preferred that the support produced be substantially free of alumina.

Calcination of the cobalt impregnated support preferably is conducted in air at temperatures in the range of about 700° C. to about 1000° C., preferably about 800° C. to about 900° C.

Typically the support will have a surface area in the range of from about 5 m²/g to about 40 m²/g and preferably from 10 m²/g to 30 m²/g. Pore volumes range from about 0.2 cc/g to about 0.5 cc/g and preferably from 0.3 cc/g to 0.4 cc/g.

In preparing the catalyst the cobalt and rhenium promoter are composited with the support by any of a variety of techniques well known to those skilled in the art, including impregnation (either co-impregnation with promoters or serial impregnation—either by spray drying or by the incipient wetness techniques). Since a preferred catalyst for fixed bed Fischer-Tropsch processes is one wherein the catalytic metals are present in the outer portion of the catalyst particle, i.e., in a layer no more than 250 microns deep, preferably no more than 200 microns deep, a preferred method of preparing the catalyst is the spray method which is described in U.S. Pat. No. 5,140,050, incorporated herein by reference or in EP 0 266 898, incorporated herein by reference. For slurry Fischer-Tropsch processes, catalysts are preferably made by incipient wetness impregnation of spray-dried supports. When using the incipient wetness impregnation technique, organic impregnation aids are optionally employed. Such aids are described in U.S. Pat. No. 5,856,260, U.S. Pat. No. 5,856,261 and U.S. Pat. No. 5,863,856, all incorporated herein by reference.

The amount of cobalt present in the catalyst will be in the range of 2 to 40 wt % and preferably 10 to 25 wt % while the rhenium will be present in weight ratios of about 1/20 to 1/10 of the weight of cobalt.

By selecting the appropriate Fischer-Tropsch reaction conditions, the appropriate catalyst, or both as described above not only is the amount of 700° F.+waxy product formed favored but the product contains a greater amount of higher molecular weight material. The 700° F.+fraction of the preferred waxy product will have greater than about 15 wt % of hydrocarbons boiling in the 850° F.-1050° F. (454° C.-565° C.) range.

A 450° F.+(232° C.+) cut of the waxy product is separated from other hydrocarbons produced in the Fischer-Tropsch process and then is catalytically hydroisomerized. Suitable hydroisomerization catalysts typically include a hydrogenating metal component such as a Group VI or Group VIII metal or mixture thereof on a refractory metal oxide support, preferably a zeolite support. The catalyst typically contains from about 0.1 wt % to about 5 wt % metal. Examples of such catalysts include a noble metal, e.g., Pt on ZSM-23, ZSM-35, ZSM-48, ZSM-57 and ZSM-22.

A preferred catalyst is Pt on ZSM-48. The preferred preparation of ZSM-48 is disclosed in U.S. Pat. No. 5,075,269 incorporated herein by reference. The Pt is deposited on the ZSM-48 by techniques well known in the art such as impregnation, either dry or by incipient wetness techniques.

Isomerization is conducted under conditions of temperatures between about 500° F. (260° C.) to about 900° F. (482° C.), preferably 550° F. (288° C.) to 725° F. (385° C.), pressures of 1 to 10,000 psi H₂, preferably 100 to 2,500 psi H₂, hydrogen gas rates of 50 to 3,500 SCF/bbl, and a space velocity in the range of 0.25 to 5 v/v/hr, preferably 0.5 to 3 v/v/hr.

Following isomerization, the isomerate is distilled into a distillate cut and a lube cut. For the purposes herein, the lube cut is that fraction boiling above about 700° F. (371° C.).

The following examples illustrate the more salient and preferred features of the invention.

### EXAMPLE 1

A syngas feed having hydrogen and carbon monoxide partial pressures of 184 and 88 psig respectively was reacted in a slurry bubble column pilot plant containing a supported cobalt-rhenium catalyst. During the course of the run the reactor temperature was increased from 410° F. (210° C.) to 430° F. (221.1° C.). The process conditions and product characterizations are given in Table 1. The catalyst was prepared by spray drying a slurry of 34.4 parts (by weight) of fumed TiO₂, 8.8 parts alumina chlorohydrol sol (containing 23.5 wt % Al₂O₃), 0.6 parts silica sol (35 wt % SiO₂) and 56.2 parts water at a rate of about 13 lb/minute through a 9 inch wheel atomizer spinning at 10,000 rpm. The spray drying chamber was operated at an air inlet temperature of about 285° C. and an outlet temperature of about 120° C.

The spray dried support was calcined in a rotary calcined at 1010° C. The support was impregnated with an aqueous solution of cobalt nitrate and perrhenic acid and calcined in air at 454° C. A second impregnation and calcination was applied to produce a final catalyst containing 11.3% Co and 1.09% Re. The catalyst was reduced in hydrogen at 371° C. and transferred under inert gas to the slurry bubble column reactor.

TABLE 1

| | Days on Syngas | | | | |
|---|---|---|---|---|---|
| | 7.2 | 9.3 | 44.3 | 70.4 | 80.4 |
| Inlet Superficial | 17.1 | 17.0 | 17.0 | 17.3 | 17.5 |
| Velocity (cm/sec) | | | | | |
| CO Conversion (%) | 49.9 | 49.6 | 42.9 | 48.6 | 42.7 |
| CH4 Selectivity (%) | 4.99 | 4.97 | 5.98 | 7.39 | 8.52 |
| Gas Hourly Space | 11680 | 11620 | 11758 | 11774 | 12061 |
| Velocity (1/hour) | | | | | |
| Reactor Temp (° F.) | 412 | 412 | 412 | 430 | 430 |

Boiling Point Distributions in Weight Percent

| | | | | | |
|---|---|---|---|---|---|
| C1 | 5.58% | 5.52% | 6.49% | 8.13% | 9.37% |
| C2 | 0.63% | 0.60% | 0.58% | 0.68% | 0.77% |
| C3-C4 | 4.88% | 4.82% | 4.47% | 4.84% | 5.54% |
| C5-320° F. | 16.54% | 16.03% | 15.75% | 19.12% | 21.20% |
| 320-500° F. | 12.55% | 12.14% | 13.23% | 12.49% | 15.97% |
| 500-700° F. | 18.46% | 18.98% | 19.74% | 20.84% | 20.17% |
| 700-850° F. | 14.65% | 14.94% | 15.40% | 15.01% | 13.25% |
| 850-1050° F. | 17.26% | 16.80% | 17.08% | 14.08% | 11.03% |
| 1050° F.+ | 9.45% | 10.18% | 7.25% | 4.80% | 2.69% |
| Total | 100.00% | 100.00% | 100.00% | 100.00% | 100.00% |
| 700+° F. | 41.36% | 41.92% | 39.73% | 33.90% | 26.97% |
| lbs 700° F.+/100 lbs | 20.8 | 21.1 | 20.0 | 17.1 | 13.6 |
| CO converted | | | | | |

As can be seen, the lower reactor temperature favored the formation of 700° F.+material. Over 20 lbs 700° F.+per 100 lbs CO converted were obtained by operating the catalyst at 412° F. rather than 430° F.

### EXAMPLE 2

A syngas feed having hydrogen and carbon monoxide partial pressures of 188 and 88 psig respectively were reacted at 410° F. (210° C.) over a catalyst comprising cobalt on a support comprising primarily titania and a minor amount of cobalt aluminate. Test conditions and product characterizations are listed in Table 2. The catalyst in this instance was prepared using a titania support prepared by spray drying as in Example 1. The spray-dried support was calcined in air in a rotary calciner at 732° F. (389° C.). The calcined support (95 parts by weight) was mixed in a V-blender with an aqueous solution of cobalt nitrate, made from 41.5 parts by weight of cobalt nitrate hexahydrate and 18.0 parts of water and the resulting product was calcined in air in a rotary calciner at 454° C. resulting in cobalt nitrate being decomposed to cobalt oxide. The product was recalcined at 870° C. which converted the cobalt oxide to cobalt aluminate and cobalt titanate. The blue-green colored final product had 5.9 wt % Co, 1.02 Co/Al atomic ratio, 94% of the TiO₂ in the rutile form, 21 m2/g surface area, and 0.31 cc/g water pore volume. This cobalt-modified support was impregnated with cobalt nitrate and perrhenic acid to form a catalyst as follows. An impregnation solution was prepared by mixing 74.0 parts cobalt nitrate hexahydrate, 1.8 parts perrhenic acid (containing 53.5 wt % Re), 5.6 parts malonic acid, and 18.6 parts water and heating the mixture to 43° C. to form a solution. By weight, 57.6 parts of this solution were added to 120 parts of cobalt-modified titania support in a V-blender mixer. The product was calcined in air in a rotary calciner at 454° C. The calcined product was impregnated a second time using the same impregnation solution, with 53 parts being added to 128 parts catalyst, and then calcined by the same procedure. The final catalyst contained 15.2% Co and 0.68% Re. The catalyst was reduced in hydrogen at 371° C. and transferred under inert gas to the slurry bubble column reactor.

TABLE 2

| | Days on Syngas | | | | | | |
|---|---|---|---|---|---|---|---|
| | 14.5 | 25.5 | 37.5 | 64.5 | 91.5 | 115.5 | 151.5 |
| Inlet Superficial | 17.5 | 17.4 | 17.3 | 13.8 | 11.0 | 8.3 | 8.3 |
| Velocity (cm/sec) | | | | | | | |
| CO Conversion (%) | 80.5 | 83.9 | 80.9 | 80.5 | 74.4 | 74.2 | 69.1 |
| CH4 Selectivity (%) | 4.44 | 4.46 | 4.58 | 4.65 | 5.22 | 3.48 | 4.28 |
| Gas Hourly Space | 10080 | 10165 | 10130 | 8152 | 6643 | 4821 | 4776 |
| Velocity (1/hour) | | | | | | | |
| Reactor Temp. (° F.) | 410 | 410 | 410 | 410 | 410 | 410 | 410 |

Boiling Point Distributions in Weight Percent

| | | | | | | | |
|---|---|---|---|---|---|---|---|
| C1 | 5.20 | 4.69 | 4.80 | 5.78 | 8.13 | 4.45 | 4.96 |
| C2 | 0.47 | 0.38 | 0.35 | 0.46 | 0.68 | 1.14 | 1.34 |
| C3-C4 | 3.90 | 4.50 | 3.04 | 3.71 | 4.84 | 8.54 | 8.93 |
| C5-320° F. | 18.26 | 15.65 | 17.24 | 18.43 | 14.15 | 20.83 | 21.77 |
| 320-500° F. | 9.15 | 10.09 | 9.78 | 10.11 | 9.77 | 7.09 | 8.08 |
| 500-700° F. | 17.81 | 17.41 | 17.82 | 14.89 | 16.49 | 14.21 | 13.00 |
| 700-850° F. | 16.07 | 15.82 | 15.88 | 15.40 | 15.82 | 13.42 | 12.09 |
| 850-1050° F. | 19.03 | 19.48 | 19.43 | 19.89 | 20.18 | 18.14 | 16.02 |
| 1050° F.+ | 10.10 | 11.97 | 11.65 | 11.33 | 14.19 | 12.18 | 13.80 |
| Total | 100.00 | 100.00 | 100.00 | 100.00 | 100.00 | 100.00 | 100.00 |
| 700°+ F. | 45.21 | 47.27 | 46.96 | 46.62 | 50.18 | 43.74 | 41.92 |
| lbs 700° F.+/100 lbs | 22.8 | 23.8 | 23.1 | 23.5 | 25.3 | 22.0 | 21.1 |
| CO converted | | | | | | | |

As can be seen the catalyst composition also impacts the amount of 700° F.+material produced per 100 lbs of CO converted. Values of about 21 to 25 are obtained by using the preferred catalyst at lower temperature.

### EXAMPLE 3

A 450° F.+(232° C.+) cut of a Fischer-Tropsch product containing 27 lbs of 700° F.+material per 100 lbs of CO converted (an alpha of 0.94) was hydroisomerized over a Pt/ZSM-48 catalyst with aluminum binder. The hydrogen form of ZSM-48 was prepared according to U.S. Pat. No. 5,075,269. The Pt component was added by impregnation followed by calcination and reduction. The hydroisomerization conditions were: temperature 622° F. (328° C.), 250 psig H₂, 2500 SCF/bbl H₂, 1 LHSV. The resulting hydroisomerate was distilled to produce a lube base oil having an initial cut point of about 950° F. (510° C.). This 950° F.+lube base oil has the properties shown in Table 3.

TABLE 3

| Viscosity @ | Viscosity @ | | Pour Point, | Cloud Point, |
|---|---|---|---|---|
| 100° C., CSt | 40° C., cSt | VI | ° C. | ° C. |
| 15.135 | 95.517 | 167.1 | −1 | 25.9 |

### EXAMPLE 4

A 450° F.+cut from a Fischer-Tropsch product containing 24 lbs of 700° F.+material per 100 lbs of CO converted (an alpha of 0.93) was hydroisomerized over a Pt/ZSM-48 catalyst described in Example 3.

The hydroisomerization conditions were as follows: temperature 587° F. (308° C.), 250 psig H₂, 2500 SCF/bbl, 1 LHSV. The hydroisomerate was then distilled to recover a 950° F.+(510° C.+) fraction. This 950° F.+fraction was subsequently hydroisomerized over a Pt/ZSM-48 catalyst as described above at 614° F. (323° C.), 250 psig H₂, 2500 SCF/bbl H₂, 1 LHSV. The resulting isomerate was distilled to produce a lube base oil having an initial cut point of about 915° F. (491° C.). The properties of the 915° F.+fraction are given in Table 3 below:

TABLE 4

| Viscosity @ | Viscosity @ | | Pour Point, | Cloud Point, |
|---|---|---|---|---|
| 100° C., cSt | 40° C., cSt | VI | ° C. | ° C. |
| 13.063 | 85.819 | 152.2 | −32 | 7.1 |

1. A method for producing high viscosity lube base oils from synthesis gas comprising
converting the synthesis gas under Fischer-Tropsch reaction conditions sufficient to produce hydrocarbon products containing greater than 20 lbs of 700° F.+product per 100 lbs of CO converted;
separating a 450° F.+cut from the hydrocarbon products;
catalytically hydroisomerizing the separated 450° F.+cut to obtain a hydroisomerized product; and
distilling the hydroisomerized product to provide a distillate cut and a high viscosity lube base oil cut.

2. The method of claim 1 wherein the synthesis gas is converted under reaction conditions including reaction temperatures no greater than 430° F.

3. The method of claim 1 wherein the synthesis gas is converted in the presence of catalyst comprising Co and Re on a support comprising titania and cobalt aluminate and wherein the Co/Al atomic ratio is greater than 0.25.

4. The method of claim 2 or 3 wherein the hydrocarbon products are hydroisomerized in the presence of a catalyst comprising a metal hydrogenating component on a refractory oxide support.

5. The method of claim 4 wherein the metal is selected from the group consisting of Group VI metals, Group VIII metals and mixtures thereof and wherein the refractory oxide support is selected from the group consisting of ZSM-23, ZSM-35, ZSM-48, ZSM-57 and ZSM-22.

6. The method of claim 5 wherein the metal is Pt and the support is ZSM-48.

7. The method of claim 5 including catalytically or solvent dewaxing the high viscosity lube base oil cut to lower the pour point.
